(19) United States
(12) Patent Application Publication
(10) Pub. No.: US 2015/0316514 A1
(43) Pub. Date: Nov. 5, 2015

(54) SIGNAL PROCESSING DEVICE, ULTRASOUND DIAGNOSTIC APPARATUS, AND SIGNAL PROCESSING METHOD

(71) Applicant: KONICA MINOLTA, INC., Tokyo (JP)

(72) Inventor: Yoshihiro TAKEDA, Tokyo (JP)

(73) Assignee: KONICA MINOLTA, INC., Tokyo (JP)

(21) Appl. No.: 14/681,629

(22) Filed: Apr. 8, 2015

(30) Foreign Application Priority Data
May 2, 2014 (JP) 2014-094935

(51) Int. Cl. G01N 29/50; G01N 29/24; A61B 8/08

(52) U.S. Cl. CPC G01N 29/50; A61B 8/485; G01N 29/24; G01N 2291/0289; G01N 2291/02827; G01N 2291/044

(57) ABSTRACT

A signal processing device which uses a first signal waveform of ultrasound reflected from an object in a first compressed state and a second signal waveform of ultrasound reflected from the object in a second compressed state and calculates an elasticity of the object affected by a change in a compressed state includes: a phase difference calculation unit which calculates a phase difference component in each time between the first signal waveform and the second signal waveform; a correlation calculation unit which calculates an elasticity pertaining to a difference in angular frequencies between the first signal waveform and the second signal waveform and an initial phase difference according to a correlation between the each time and the phase difference component in the each time; and an elasticity calculation unit which calculates the elasticity on the basis of the difference in angular frequencies.

The entire disclosure of Japanese Patent Application No. 2014-094935 filed on May 2, 2014 including description, claims, drawings, and abstract are incorporated herein by reference in its entirety.

### BACKGROUND OF THE INVENTION

1. Field of the Invention

The present invention relates to a signal processing device, an ultrasound diagnostic apparatus, and a signal processing method.

2. Description of the Related Art

There is provided, in the related art, an ultrasound diagnostic apparatus that irradiates the interior of an object with ultrasound and examines an internal structure by receiving and analyzing a reflected wave of the ultrasound. An ultrasound diagnosis can be performed on the object in non-destructive, non-invasive manners and thus used for a variety of purposes such as an examination for medical purposes and an examination inside a building.

The ultrasound is mainly reflected from a discontinuous surface on which the material and state of the interior vary. When the material and state of the interior are known, one can obtain a sufficient result by just acquiring information on the reflected position. On the other hand, there is a technique which determines the material and state by measuring hardness of the interior when the material and state of the interior cannot be confirmed. In this technique, the material and state are determined by a physical parameter such as a modulus of elasticity obtained on the basis of an elasticity of the interior that is calculated.

There is employed, as a method of calculating the elasticity, a method which compresses a reflected waveform obtained in normal measurement or stretches a reflected waveform obtained by compressing the object with a predetermined pressure to find a cross-correlation with the other waveform, detects a case that results in a high cross-correlation, and measures the elasticity on the basis of the relationship between a compression rate or an stretch rate and the pressure.

However, the position of the material itself shifts, namely undergoes parallel displacement, when calculating the elasticity of material of the interior on the basis of pressing force so that the waveform needs to be further compressed or stretched while moving one waveform relative to the other waveform to perform positioning when finding the cross-correlation between the waveforms. JP 2004-57652 A discloses a technique which finds an optimal solution by mapping all displacement candidates, for example. On the other hand, JP 2008-126079 A discloses a technique which finds the solution by first finding the displacement and then making it asymptotic to an optimal elasticity rate by successively performing calculation.

The method disclosed in JP 2004-57652 A however requires a resource that meets the enormous throughput using a CPU and a memory. Moreover, the positioning is performed first in the technique of the related art so that, when the positioning is not performed accurately, the compression rate is not calculated accurately due to a deviation generated in the calculation.

### SUMMARY OF THE INVENTION

An object of the present invention is to provide a signal processing device, an ultrasound diagnostic apparatus, and a signal processing method which can reduce a calculation error of the elasticity by easy processing.

To achieve the abovementioned object, according to an aspect, a signal processing device which uses a first signal waveform of ultrasound reflected from an object in a first compressed state and a second signal waveform of ultrasound reflected from the object in a second compressed state and calculates an elasticity of the object affected by a change in a compressed state, reflecting one aspect of the present invention, comprises: a phase difference calculation unit which calculates a phase difference component in each time between the first signal waveform and the second signal waveform; a correlation calculation unit which calculates an elasticity pertaining to a difference in angular frequencies between the first signal waveform and the second signal waveform and an initial phase difference according to a correlation between the each time and the phase difference component in the each time; and an elasticity calculation unit which calculates the elasticity on the basis of the difference in angular frequencies.

To achieve the abovementioned object, according to an aspect, a signal processing device which uses a first signal waveform of ultrasound reflected from an object in a first compressed state and a second signal waveform of ultrasound reflected from the object in a second compressed state and calculates an elasticity of the object affected by a change in a compressed state, reflecting one aspect of the present invention, comprises: an approximated waveform generation unit which compresses or stretches and phase-shifts the second signal waveform by an elasticity and an initial phase difference related to a difference in angular frequencies being set and generates an approximated signal waveform approximated to the first signal waveform; a phase difference calculation unit which calculates a phase difference component in each time between the first signal waveform and the approximated signal waveform; a correlation calculation unit which calculates the elasticity and the initial phase difference between the first signal waveform and the approximated signal waveform according to a correlation between the each time and the phase difference component in the each time; a repeat determination unit which updates the approximated signal waveform by compressing or stretching and phase-shifting the approximated signal waveform by using the elasticity and the initial phase difference calculated by the correlation calculation unit until a predetermined condition is satisfied, and uses the updated approximated waveform and the first signal waveform to repeat processing by the phase difference calculation unit and the correlation calculation unit; and an elasticity calculation unit which calculates the elasticity on the basis of a cumulative value of the elasticity related to the compression repeatedly performed.

According to the signal processing device of Item. 2, the signal processing device of Item. 3 preferably includes a recovery rate calculation unit which calculates a recovery rate indicating a correlation between the first signal waveform and the approximated signal waveform when the predetermined condition is satisfied.

According to the signal processing device of Item. 3, the signal processing device of Item. 4 preferably includes a display unit and a display control unit which causes the display unit to display the elasticity and calculation accuracy of the elasticity based on the recovery rate.

According to the signal processing device of Item. 3 or 4, the signal processing device of Item. 5 preferably includes a noise removal unit which smoothes a space distribution of the elasticity calculated, where the noise removal unit calculates magnitude of the smoothed elasticity in each spatial position by a weighted average of magnitude of the elasticity in each of a plurality of positions within a predetermined range corresponding to the spatial position, and a weight of the weighted average is determined on the basis of magnitude of the recovery rate in each of the plurality of positions.

According to the signal processing device of any one of Items. 2 to 5, in the signal processing device of Item. 6, the phase difference calculation unit preferably changes a width of the time pertaining to the calculated phase difference component according to at least one of the elasticity and the initial phase difference between the first signal waveform and the approximated signal waveform.

According to the signal processing device of any one of Items. 2 to 6, in the signal processing device of Item. 7, the repeat determination unit preferably sets, as the predetermined condition, a case where the elasticity between the first signal waveform and the approximated signal waveform equals a predetermined reference value or smaller.

According to the signal processing device of any one of Items. 1 to 7, in the signal processing device of Item. 8, the correlation calculation unit preferably calculates an optimal value of the elasticity and the initial phase difference by a least squares method using the elasticity and the initial phase difference as parameters.

An ultrasound diagnostic apparatus of Item. 9 preferably comprises: an ultrasound probe which transmits/receives ultrasound; and the signal processing device of any one of Items. 1 to 8.

To achieve the abovementioned object, according to an aspect, a signal processing method which uses a first signal waveform of ultrasound reflected from an object in a first compressed state and a second signal waveform of ultrasound reflected from the object in a second compressed state and calculates an elasticity of the object affected by a change in a compressed state, reflecting one aspect of the present invention, comprises: a phase difference calculation step which calculates a phase difference component in each time between the first signal waveform and the second signal waveform; a correlation calculation step which calculates an elasticity pertaining to a difference in angular frequencies between the first signal waveform and the second signal waveform and an initial phase difference according to a correlation between the each time and the phase difference component in the each time; and an elasticity calculation step which calculates the elasticity on the basis of the elasticity.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a general view illustrating an ultrasound diagnostic apparatus of an embodiment of the present invention;

FIG. 2 is a block diagram illustrating an internal configuration of the ultrasound diagnostic apparatus;

FIGS. 3A and 3B are diagrams illustrating how an elasticity is measured;

FIG. 4 is a diagram illustrating a flow of calculating and displaying the elasticity;

FIG. 5 is a flowchart illustrating a control procedure of elasticity measurement/display processing;

FIG. 6 is a flowchart illustrating a control procedure of elasticity calculation processing; and

FIGS. 7A and 7B are diagrams each illustrating a display example of an elasticity display image.

### DESCRIPTION OF THE PREFERRED EMBODIMENTS

Hereinafter, an embodiment of the present invention will be described with reference to the drawings. However, the scope of the invention is not limited to the illustrated examples.

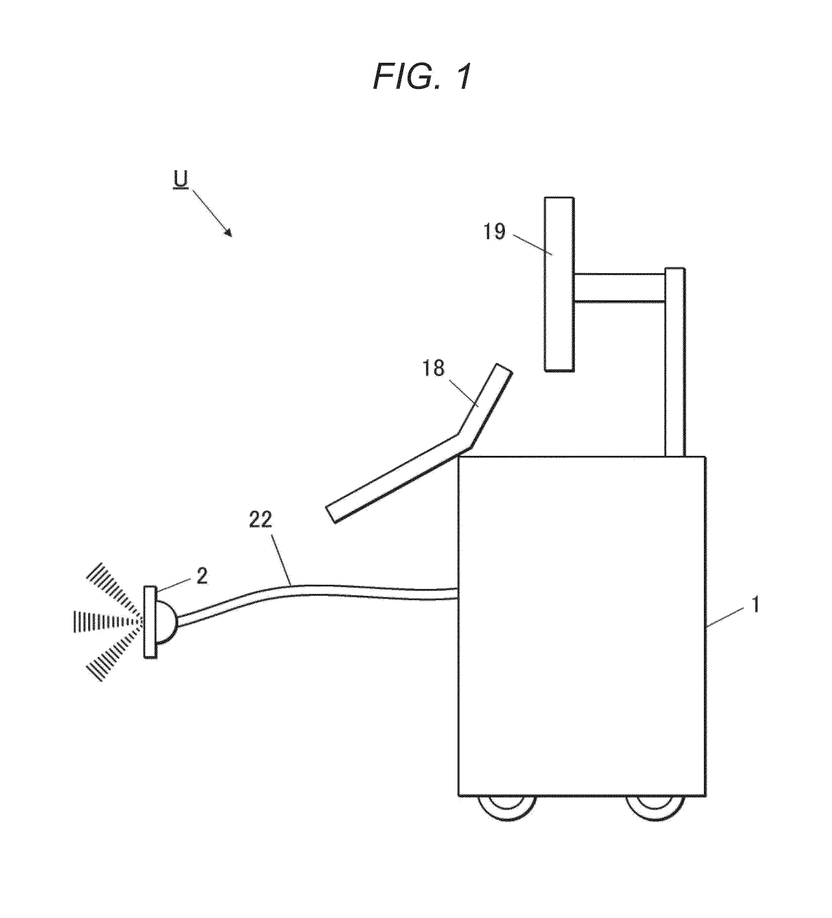

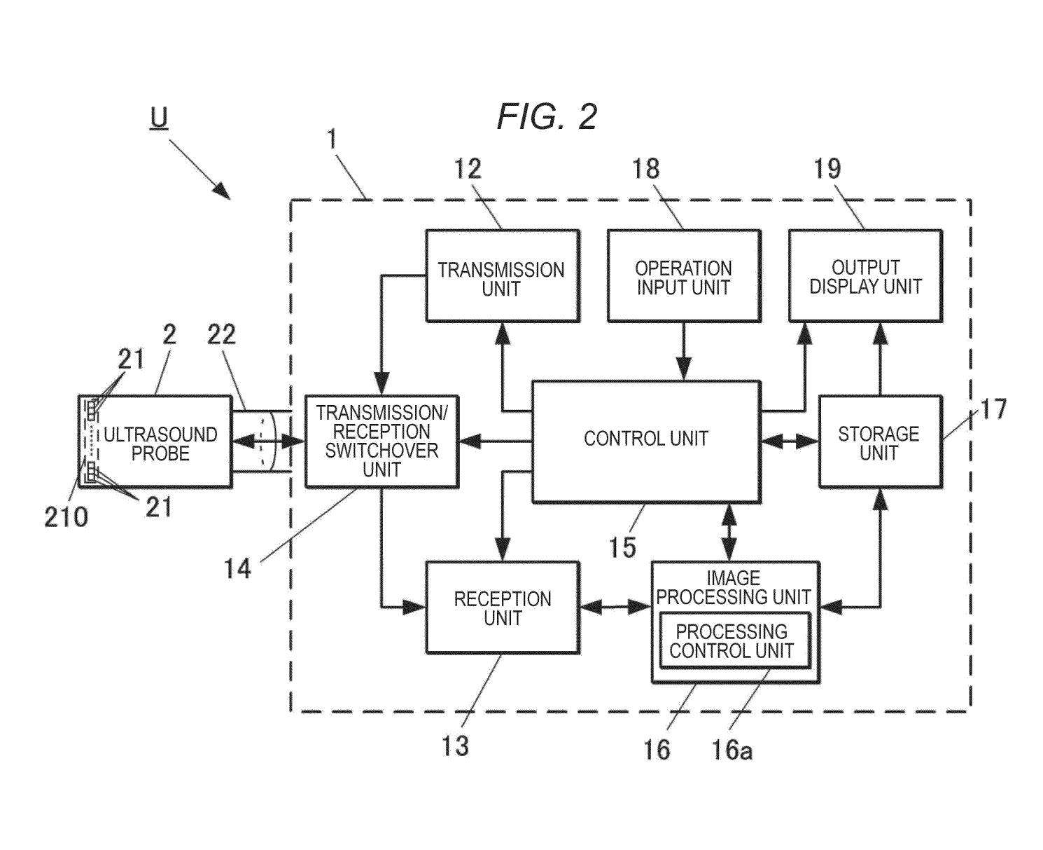

FIG. 1 is a general view of an ultrasound diagnostic apparatus of the present embodiment. FIG. 2 is a block diagram illustrating an internal configuration of the ultrasound diagnostic apparatus U.

As illustrated in FIG. 1, the ultrasound diagnostic apparatus U includes an ultrasound diagnostic apparatus body 1 and an ultrasound probe 2 connected to the ultrasound diagnostic apparatus body 1 through a cable 22. The ultrasound diagnostic apparatus body 1 is provided with an operation input unit 18 and an output display unit 19. A control unit 15 of the ultrasound diagnostic apparatus body 1 causes the ultrasound probe 2 to output ultrasound by outputting a drive signal to the ultrasound probe 2 on the basis of an input operation externally performed on an input device such as a keyboard or mouse of the operation input unit 18, performs various processings by acquiring a reception signal corresponding to the ultrasound received from the ultrasound probe 2, and causes a liquid crystal screen of the output display unit 19 to display a result as needed.

As illustrated in FIG. 2, the ultrasound diagnostic apparatus body 1 includes a transmission unit 12, a reception unit 13, a transmission/reception switchover unit 14, the control unit 15, an image processing unit 16, a storage unit 17, the operation input unit 18, and the output display unit 19 (display unit).

The transmission unit 12 outputs a pulse signal to be supplied to the ultrasound probe 2 according to a control signal input from the control unit 15, and causes the ultrasound probe 2 to generate ultrasound. The transmission unit 12 includes a clock generation circuit, a pulse generation circuit, a pulse width setting unit, and a delay circuit, for example. The clock generation circuit is a circuit which generates a clock signal determining a transmission timing and a transmission frequency of the pulse signal. The pulse generation circuit is a circuit which generates a bipolar square wave pulse with a predetermined voltage amplitude at a predetermined cycle. The pulse width setting unit sets a pulse width of the square wave pulse output from the pulse generation circuit. The square wave pulse generated by the pulse generation circuit is separated into a wiring path different for each transducer 21 of the ultrasound probe 2 before or after being input to the pulse width setting unit. The delay circuit is a circuit which delays the output of the generated square wave pulse by a delay time set to each wiring path according to the timing at which the pulse is transmitted to each transducer 21.

The reception unit 13 is a circuit which acquires the reception signal input from the ultrasound probe 2 under control of the control unit 15. The reception unit 13 includes an amplifier, an A/D conversion circuit, and a phasing/adding circuit, for example. The amplifier is a circuit which amplifies, at a predetermined amplification factor, the reception signal corresponding to the ultrasound received from each transducer 21 of the ultrasound probe 2. The A/D conversion circuit is a circuit which converts the amplified reception signal into digital data at a predetermined sampling frequency. The sampling frequency requires that a Nyquist frequency be higher than a reception frequency (to be described) and is 60 MHz, for example. The phasing/adding circuit is a circuit which regulates the time phase of the A/D-converted reception signal by applying a delay time to each wiring path corresponding to each transducer 21, adds the signals (phasing and adding), and generates sound ray data.

Under control of the control unit 15, the transmission/reception switchover unit 14 performs a switchover operation of causing the transmission unit 12 to transmit the drive signal to the transducer 21 when the ultrasound is emitted from the transducer 21 as well as causing the reception unit 13 to output the reception signal when a signal corresponding to the ultrasound emitted by the transducer 21 is acquired.

The control unit 15 includes a CPU (Central Processing Unit), an HDD (Hard Disk Drive), and a RAM (Random Access Memory). The CPU reads various programs stored in the HDD, loads them to the RAM, and performs integrated control of an operation of each unit in the ultrasound diagnostic apparatus U according to the programs. The HDD stores a control program and various processing programs used to operate the ultrasound diagnostic apparatus U as well as various setting data. These programs and setting data may also be stored in an auxiliary storage using a non-volatile memory such as a flash memory in a readable/writable manner in addition to being stored in the HDD. The RAM being a volatile memory such as an SRAM and a DRAM provides memory space for work performed by the CPU and stores temporary data.

The image processing unit 16 has a processing control unit 16a which includes, separately from the CPU of the control unit 15, a CPU and a RAM performing computational processing to create a diagnostic image based on received data of the ultrasound. The diagnostic image includes image data, a series of video data thereof, and still image data of a snapshot that are displayed near real-time on the output display unit 19. Moreover, a distribution of the elasticity (elasticity value) inside the object can be calculated and displayed/output as the still image data of the snapshot.

The image processing unit 16 makes up a signal processing device, while the processing control unit 16a makes up a phase difference calculation unit, a correlation calculation unit, an elasticity calculation unit, an approximated waveform generation unit, a repeat determination unit, a recovery rate calculation unit, a display control unit, and a noise removal unit. The processing control unit 16a also functions as a control unit of the signal processing device that executes various processings related to a signal processing method of the present invention including a phase difference calculation step, a correlation calculation step, and an elasticity calculation step.

Note that the CPU 15 may be adapted to perform these computational processings performed by the processing control unit 16a.

The storage unit 17 is a volatile memory such as a DRAM (Dynamic Random Access Memory). The storage unit 17 may instead be one of various non-volatile memories capable of performing high-speed rewrite. The storage unit 17 stores diagnostic image data frame by frame, the image being processed by the image processing unit 16 and used for real-time display or display according thereto. Ultrasound diagnostic image data stored in the storage unit 17 is read under control of the control unit 15 to be transmitted to the output display unit 19 or output to the outside of the ultrasound diagnostic apparatus U through a communication unit not shown. When the output display unit 19 employs a television mode as a display mode, a DSC (Digital Signal Converter) may be provided between the storage unit 17 and the output display unit 19 so that the image data is output after a scan format is converted.

The operation input unit 18 includes a push button switch, a keyboard, a mouse or a track ball, or a combination of these that converts an input operation of a user into an operation signal, and inputs the signal to the ultrasound diagnostic apparatus body 1.

The output display unit 19 includes a display screen and a drive unit thereof, the display screen adopting any one of various display types including an LCD (Liquid Crystal Display), an organic EL (Electro-Luminescent) display, an inorganic EL display, a plasma display, and a CRT (Cathode Ray Tube) display. The output display unit 19 generates a drive signal of the display screen (each display pixel) according to a control signal output from the CPU 15 or the image data generated by the image processing unit 16, and displays on the display screen a menu and a status pertaining to an ultrasound diagnosis as well as measurement data based on the ultrasound received.

These operation input unit 18 and output display unit 19 may be provided integrally with a casing of the ultrasound diagnostic apparatus body 1 or attached externally through a USB cable or an HDMI cable (registered trademark: HDMI). Alternatively, peripherals for operation and display used in the related art may be connected to an operation input terminal and a display output terminal of the ultrasound diagnostic apparatus body 1, when provided.

The ultrasound probe 2 functions as an acoustic sensor which emits (sends out) ultrasound (approximately 1 to 30 MHz in this case) to the object such as a living body by oscillating the ultrasound as well as receives a reflected wave (echo) of the emitted ultrasound reflected from the object and converts it into an electric signal. The ultrasound probe 2 includes a transducer array 210 and the cable 22, the transducer array being an array of the plurality of transducers 21 transmitting/receiving ultrasound. One end of the cable 22 has a connector (not shown) to be connected to the ultrasound diagnostic apparatus body 1 so that the ultrasound probe 2 can be attached/detached to/from the ultrasound diagnostic apparatus body 1 by the cable 22. The user brings a surface of the ultrasound probe 2 from which the ultrasound is transmitted/received into contact with the object with a predetermined pressure, operates the ultrasound diagnostic apparatus U, and performs an ultrasound diagnosis, the surface corresponding to a surface facing a direction in which the ultrasound is emitted from the transducer array 210.

The ultrasound probe 2 can also be configured to include a pressure sensor to measure the pressure applied to the object from the ultrasound probe 2 and output it to the control unit 15. Furthermore, the ultrasound probe 2 may be configured to include a motor that moves the transmission/reception surface of the ultrasound probe 2 in a direction the ultrasound is transmitted/received so that the probe can be pressed against the object with a predetermined pressure or released therefrom.

The transducer array 210 is an array of the plurality of transducers 21 including a piezoelectric element having a piezoelectric body and an electrode which is provided at both ends and at which an electrical charge appears by the deformation (expansion) of the piezoelectric body, the transducer array being a one dimensional array provided in a predetermined direction (scanning direction), for example. The ultrasound is emitted when the piezoelectric body is deformed in response to an electric field generated in each piezoelectric body by a voltage pulse (pulse signal) sequentially supplied to the transducer 21. When the ultrasound of a predetermined frequency band enters the transducer 21, the sound pressure of the ultrasound causes the piezoelectric body to change in thickness (vibrate) so that an electrical charge corresponding to the amount of change is generated and converted/output into an electrical signal corresponding to the amount of electrical charge.

Next, an operation of measuring the elasticity in the ultrasound diagnostic apparatus U of the present embodiment will be described.

The ultrasound diagnostic apparatus U of the present embodiment includes a B mode in which brightness is used to perform near real-time one-dimensional to two-dimensional display of a tomography examination, an M mode in which the Doppler effect is used to measure and display a blood flow state, and an elasticity display mode in which the elasticity of an internal structure is measured and displayed.

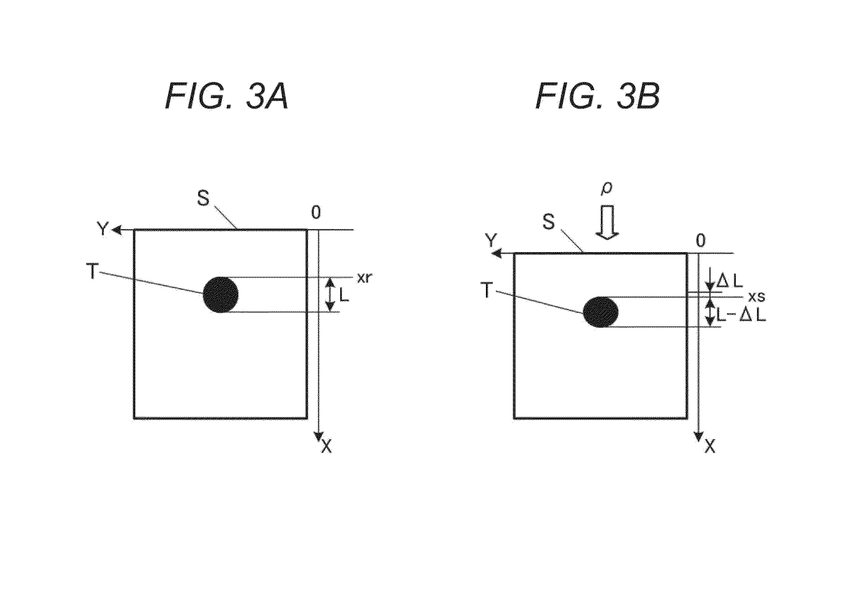

FIGS. 3A and 3B are diagrams illustrating how the elasticity is measured.

Inside an object S, in normal time, an upper end of a structure T is positioned a distance xr away from a top surface of the object S in a depth direction (X direction), the top surface being a surface in contact with a surface of the ultrasound probe 2 from which the ultrasound is emitted, as illustrated in FIG. 3A. A reference numeral L indicates the width of the structure T in the X direction. When a pressure p (stress) is applied to the structure T while the same pressure p is applied to the object S from the top surface side as illustrated in FIG. 3B, the structure T undergoes a change such that the upper end thereof is positioned at a distance xs in the X direction and that the width is L−ΔL.

As a result, an elasticity ε=ΔL/L can be found by measuring the structure T in these two states. At this time, the pressure p (stress) measured by the pressure sensor can be used to calculate and display a modulus of longitudinal elasticity (Young's modulus) E=ρ/ε.

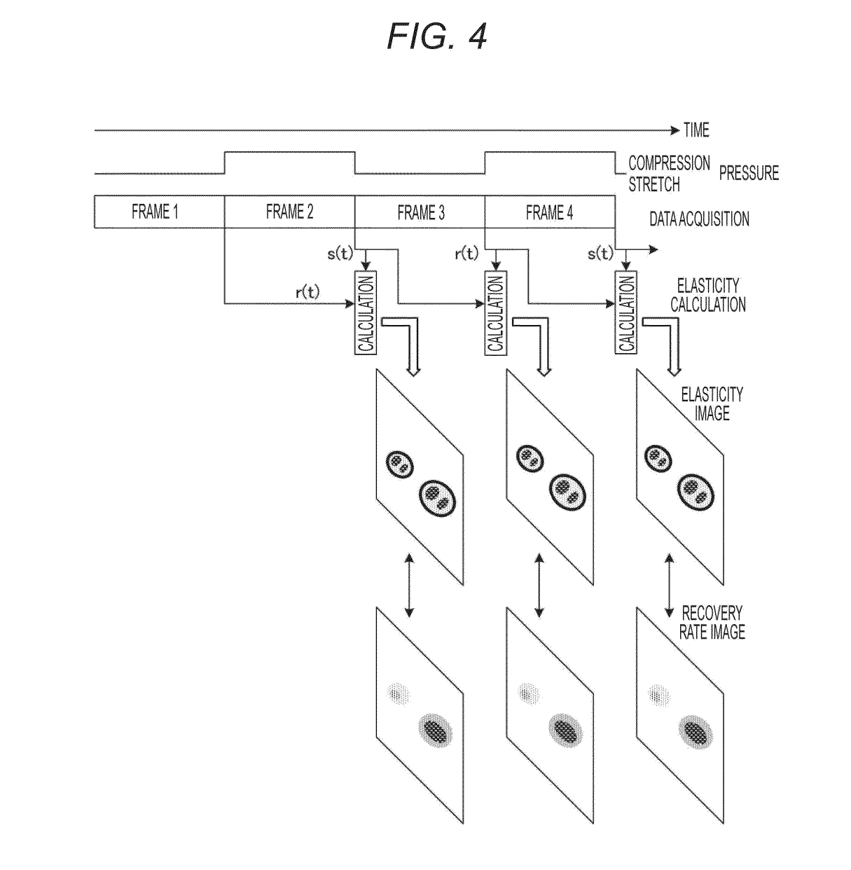

FIG. 4 is a diagram illustrating a flow of calculating and displaying the elasticity.

In the elasticity display mode, the ultrasound is transmitted/received while applying two different types of pressures to the object (changing the pressure applied by the ultrasound probe 2 against the object, for example). Here, data of each frame is acquired while alternately switching the magnitude of two types of pressures in each frame. Once two frames' worth of echo is acquired upon application of different pressures, the elasticity and a recovery rate thereof at each position are calculated on the basis of the two frames' worth of data. That is, the data in each frame is used twice to calculate the distribution of the elasticity excluding the first and last frame data (such as a frame 1). Data of the distribution of the elasticity and recovery rate calculated are then objected to processing such as smoothing and adjustment of a dynamic range for display and displayed in the output display unit 19 in color or gray scale, for example.

The recovery rate is an index indicating the accuracy of the measured value of elasticity and will be described later.

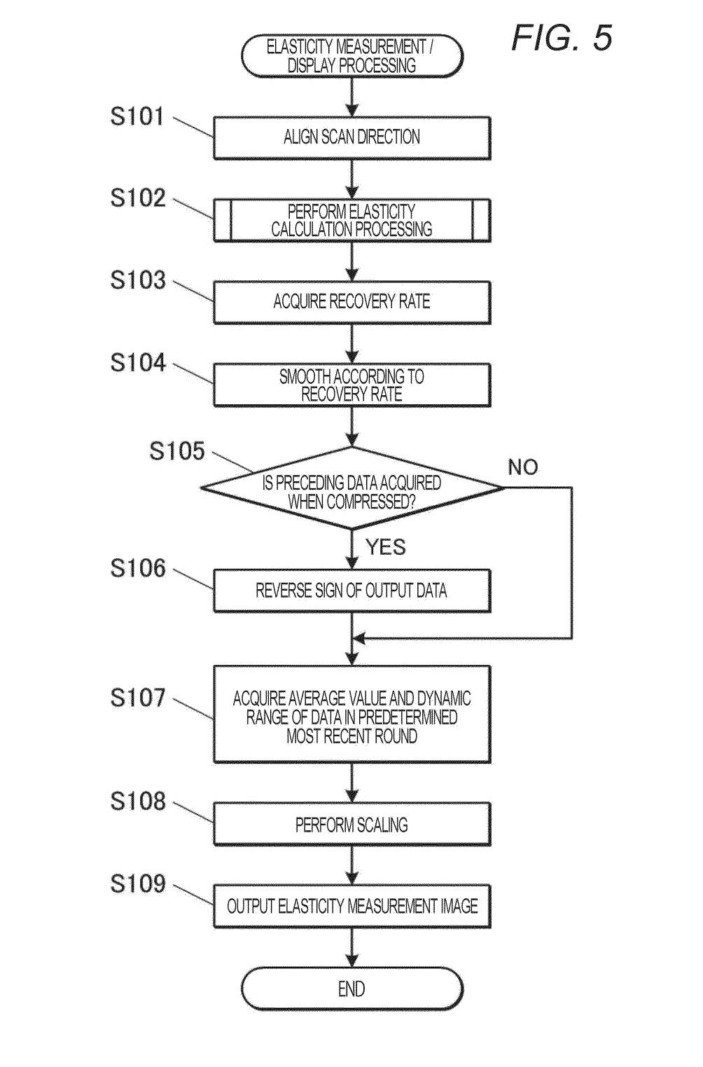

FIG. 5 is a flowchart illustrating a control procedure performed by the processing control unit 16a of the image processing unit 16 in the elasticity measurement/display processing executed in the ultrasound diagnostic apparatus U.

The elasticity measurement/display processing is executed on the basis of a control signal transmitted from the control unit 15 to the image processing unit 16 when the elasticity measurement/display mode is selected by an input operation performed by the user on the operation input unit 18 in the measurement/display processing involved in an ultrasound diagnosis.

When the elasticity measurement/display processing is started, the processing control unit 16a (CPU) of the image processing unit 16 acquires received waveform data of two scans (two frames) performed on the object with different pressures and performs alignment in the scan direction of the ultrasound probe 2 (step S101). The processing control unit 16a determines a combination of scan positions corresponding to the same position by adjustment between preset positions or pattern matching.

The processing control unit 16a uses a combination of an ultrasound waveform (stretched waveform or first signal waveform) acquired when small force is applied to the object (first compressed state) and an ultrasound waveform (compressed waveform or second signal waveform) acquired when large force is applied to the object (second compressed state) at each scan position, and calculates the elasticity resulting from a change in the compressed state (step S102). The processing control unit 16a further calculates and acquires the recovery rate related to the calculated elasticity (step S103). Processing of calculating the elasticity will be described later in detail.

The processing control unit 16a performs smoothing on each point in a two dimensional image of two rounds of scan images (two frames' worth of data) with intensity corresponding to the recovery rate (step S104). The processing control unit 16a also determines whether or not the first one of the received waveform data acquired in two rounds of scan is a compressed waveform (step S105). When the first received waveform data is determined to be the data of the compressed waveform (“YES” in step S105), the sign of the calculated value of the two dimensional image data subjected to smoothing in step S104 is reversed, or the sign of ΔL of the elasticity is reversed (step S106). The processing control unit 16a thereafter proceeds to step S107. When the first received waveform data is determined to be not the data of the compressed waveform (“NO” in step S105), the processing control unit 16a proceeds to step S107.

Upon proceeding to step S107, the processing control unit 16a acquires an average value and a dynamic range of each data of the two dimensional image data acquired in the predetermined most recent round (step S107). These values can be acquired easily by storing a predetermined number of these values in a RAM of the processing control unit 16a every time a final piece of two dimensional image data is output in each round of the elasticity measurement/display processing. The processing control unit 16a performs scaling of the two dimensional image on the basis of the values acquired in the present round as well as the average value and dynamic range acquired in the predetermined most recent round (step S108). The processing control unit 16a then causes the output display unit 19 to output, through the storage unit 17, the data of the scaled two dimensional image of the elasticity measurement (elasticity measurement image) (step S109). The processing control unit 16a now ends the elasticity measurement/display processing.

Next, the processing of calculating the elasticity performed in the ultrasound diagnostic apparatus U of the present embodiment will be described.

The ultrasound diagnostic apparatus U of the present embodiment presses the ultrasound probe 2 against the same region of the object with two different types of pressures (including zero and negative pressures (tension)) and acquires an echo of the ultrasound emitted in each of the pressured states, where the echo with the smaller pressure is acquired as an stretched waveform r(t) while the echo with the larger pressure is acquired as a compressed waveform s(t).

The stretched waveform r(t) at each data acquisition timing (elapsed time t (time)) is expressed as

r(t)=A₁(t)cos(ω₀t+φ₁(t)) (1).

In the expression, ω₀ denotes a center angular frequency of the received ultrasound, A₁(t) denotes a temporal change of an amplitude component (envelope of the received waveform), and φ₁₍ₜ₎ denotes an initial phase.

This waveform can be expressed analytically by the following complex function.

rₐ(t)=A₁(t)exp(iω₀t+φ₁(t)) (2)

As for the compressed waveform s(t), on the other hand, the echo from a predetermined structure is observed in a shorter time or shorter cycle than the stretched waveform r(t) according to the elasticity ε(namely the stretch rate that is ε<0 when compressed). Moreover, the indirect pressure on the object causes the position of the object to move from xr to xs in the interior, so that a detection timing, namely the phase, of the reflected wave changes. In a range where the elasticity ε is small (normally 5% or less, for example), an stretched waveform rₐ(t) is compressed by the amount of elasticity ε to be able to obtain an approximated waveform cₐ(t) that is approximate to the compressed waveform s(t) as expressed by expression (3).

cₐ(t)=A₁(t(1−ε)exp(iω₀t(1−ε)+φ₁(t(1−ε)) (3)

These analytic solutions (2) and (3) are used to find a phase difference Fₐ(t) (phase difference component) between the stretched waveform r(t) and the approximated waveform cₐ(t) by the following expression (4).

Fₐ(t)=Im(log(rₐ(t)cₐ*(t)))=εω₀t+δ (4)

In the expression, cₐ*(t) denotes a complex conjugate of the approximated waveform cₐ(t), and δ denotes a phase shift (initial phase difference) caused by the deviation between the distance xs and the distance xr. That is, the phase difference Fₐ(t) is a linear function where a slope is proportional to the elasticity ε and an intercept is expressed by the phase shift δ. The phase difference Fₐ(t), particularly the phase shift δ does not necessarily correspond to a value within a single cycle of the phase εω₀t that changes time dependently.

When the phase difference Fₐ(t) is used to find a phase difference F(t) between the measured stretched waveform r(t) and compressed waveform s(t), the value of an imaginary number portion in an analytic solution is the value obtained by shifting the phase of the measured value (real number portion) of each of the stretched waveform and the compressed waveform by ±90 degrees (delaying time of the angular frequency ω₀ by the phase corresponding to π/2 and 3π/2). The waveform of each of the real number portion and the imaginary number portion may be acquired as an I wave and a Q wave by objecting the received waveforms to an IQ quadrature detection, in which case the wave may be converted into a signal of an appropriate intermediate frequency.

The measured data of the compressed waveform s(t) used in this case can be replaced by the approximated waveform c(t) (approximated signal waveform) generated by stretch and shift (compression and phase shift, the compression including a negative compression rate, or stretch) corresponding to the elasticity ε and the phase shift δ being set, so that the difference between the stretched waveform r(t) and the approximated waveform c(t) is made small.

A default value of each of the elasticity ε and the phase shift δ can be set to a value obtained by calculation of the elasticity ε at an adjacent position, for example. Alternatively, the default value may be set to standard elasticity ε and phase shift δ estimated according to expected internal structure and change in applied pressure. When the calculation started with the elasticity ε and phase shift δ found at the previous position (adjacent position) does not converge to an accurate value or when the recovery rate at that position is low, the calculation of the elasticity ε and phase shift δ can be initialized to be switched to calculation based on theoretical values or can be switched to calculation using values calculated the time before last. Moreover, these default values can be set to “0” when the pressure is small enough.

The phase component can also be calculated after standardizing in advance the amplitude of the compressed waveform s(t) and stretched waveform r(t).

The phase difference F(t) is calculated by two complex functions based on the aforementioned measured values. In other words, the phase difference is obtained by finding a multiplication product of the real number portion of the stretched waveform r(t) and the imaginary number portion, the sign of which is reversed, of the approximated waveform c(t) and a multiplication product of the imaginary number portion of the stretched waveform r(t) and the real number portion of the approximated waveform c(t), and adding these multiplication results together. A deviation η remaining in the value of F(t) found by applying the elasticity ε and phase shift δ being set within a range (window) of a predetermined elapsed time t is expressed by expression (5).

η(ε,δ)=F(t)−εω₀t−δ (5)

Then, the elasticity ε and phase shift δ giving the smallest sum of squares σ of the deviation η expressed in expression (6) are calculated.

σ=Σₜη²=Σₜ(F(t)−εω₀t−δ)² (6)

In the expression, Σₜ denotes a sum of digital discrete values of the elapsed time t associated with sampling data. The elasticity ε(angular frequency difference εω₀) and phase shift δ giving the smallest sum of squares σ can be easily found by the least squares method applied to a linear function.

The calculated elasticity ε and phase shift δ represent a deviation of the elasticity ε(elasticity) and a deviation of the phase shift δ used in the approximated waveform c(t). Accordingly, the calculated values are accumulated to each of the values used in the approximated waveform c(t) so that the elasticity ε and phase shift δ can be brought closer to the accurate values by the cumulative value.

The more accurate elasticity ε and phase shift δ found in the aforementioned manner are used to generate the approximated waveform c(t) objected to stretch and shift for the second time. When the compressed waveform s(t) acquired with respect to the discrete elapsed time t is faithfully stretched on the basis of the elasticity ε, namely the stretch rate, there is generated a deviation between the shifted elapsed time t and the original elapsed time t. Accordingly, the elapsed time is determined by approximation within the rage corresponding with the original elapsed time t, or data of the approximated waveform c(t) after stretch is used to find the amplitude intensity in the original elapsed time t by interpolation. The interpolation can be performed by selecting a known interpolation method besides a primary linear interpolation.

The accurate elasticity ε and phase shift δ are found asymptotically by repeating such processing. A condition to end the repeating (predetermined condition) is determined as appropriate. The end condition may simply be a fixed number of executions, or may be a case where a deviation of the elasticity ε(elasticity, or angular frequency difference) found by the least squares method or both the deviation of the elasticity ε and the deviation of the phase shift δ is/are smaller than or equal to a reference value, for example. Moreover, an error may be output saying that it is difficult to calculate the elasticity ε and phase shift δ at the position when the deviations do not become smaller than or equal to the reference value within the fixed number of times.

At this time, the phase difference F(t) is a function that circulates in the range of width 2π (such as −π<F(t)≦π) according to the elapsed time t, and when the range of the elapsed time t (time width) is too wide for the product of the elasticity ε being the slope and the angular frequency ω₀ of the received ultrasound or when the timing between the range of width 2π and the range of the elapsed time t does not match due to the phase shift δ, a foldback appears in the middle of the range of the elapsed time t, namely there appears a point where the value of the phase difference jumps from the maximum value (n in this case) to the minimum value (−π in this case), whereby a correlation analysis of the linear function using the least squares method is not performed properly. Such foldback occurs less likely by setting the range of the elapsed time t narrower when the calculated elasticity ε and phase shift δ are possibly large as when the elasticity ε and phase shift δ are calculated for the first time. When the elasticity ε and the phase shift δ are calculated for the second time on, the range of the elapsed time t can be extended according to the magnitude of the elasticity ε and initial phase shift δ used in updating the approximated waveform c(t), a recovery rate R and the number of processings k, so that the number of data points used in the correlation analysis can be increased to increase an S/N ratio and increase the accuracy of the elasticity ε and the phase shift δ calculated in the end.

Moreover, the foldback occurs less likely by dropping the received angular frequency ω₀ of the ultrasound, namely the emission frequency, when the elasticity ε and the phase shift S cannot be calculated satisfactorily by the aforementioned processing.

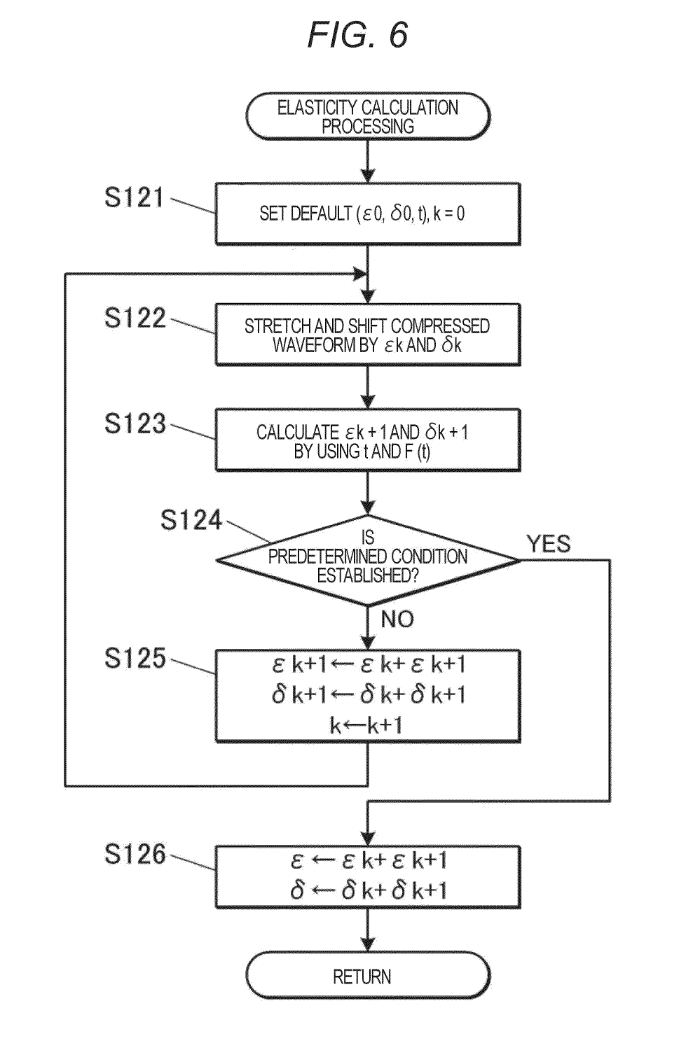

FIG. 6 is a flowchart illustrating a control procedure performed by the processing control unit 16a in the elasticity calculation processing that is called by the elasticity measurement/display processing and executed.

Once the elasticity calculation processing is called up, the processing control unit 16a sets a default value ε₀ of the elasticity and a default value δ₀ of the phase shift. The processing control unit also sets the number of processings k to “0” and sets the range of the elapsed time t for which the sum of squares σ is calculated (step S121).

The processing control unit 16a uses the elasticity εₖ and phase shift δₖ being set to stretch and shift the compressed waveform s(t) and generate data of an approximated waveform cₖ(t) (step S122). The processing control unit 16a then uses the stretched waveform r(t) and the approximated waveform cₖ(t) to calculate the phase difference F(t) at each sampling timing (elapsed time t) within the range of the elapsed time being set, and calculates for the phase difference F(t) and elapsed time t a new elasticity εₖ₊₁ and a new phase shift δₖ₊₁ by employing the least squares method (step S123).

The processing control unit 16a determines whether or not a predetermined condition is established (step S124) and, when determining the predetermined condition is not established (“NO” in step S124), adds the newly calculated elasticity εₖ₊₁ and the previous elasticity εₖ. Moreover, the processing control unit 16a adds the newly calculated phase shift δₖ₊₁ to the previous phase shift δₖ. The processing control unit 16a then adds 1 to the number of processings k (step S125) and returns to step S122.

When determining that the predetermined condition is established (“YES” in step S124), the processing control unit 16a adds the current elasticity εₖ₊₁ and the previous elasticity εₖ and makes it the value of the final elasticity ε. Moreover, the processing control unit 16a adds the current phase shift δₖ₊₁ and the previous phase shift δₖ, and makes it the final phase shift δ (step S126). The processing control unit 16a thereafter ends the elasticity calculation processing and returns to the elasticity measurement/display processing.

At this time, a cross-correlation coefficient between the measured stretched waveform r(t) and the approximated waveform cₖ(t) generated by using the elasticity ε and phase shift δ calculated in the end is the recovery rate R. That is, when the recovery rate R calculated by the processing performed in step S103 in the elasticity measurement/display processing equals “1”, the stretched waveform r(t) before compression is perfectly obtained from the compressed waveform s(t).

Two dimensional data of the elasticity acquired in the elasticity calculation processing usually contains a large amount of noise and is thus objected to smoothing in step S104 of the elasticity measurement/display processing when illustrated in the figure. The smoothing is performed, for example, such that the elasticity ε found at each point of coordinates (x, y) of a two dimensional image equals a weighted average of data of the magnitude of the elasticity at each position (a plurality of positions) within a region (predetermined range) where a straight line connecting coordinates (x−M, y−N) and coordinates (x+M, y+N) becomes a diagonal line (where each of M and N is a natural number set as appropriate). Moreover, at this time, the weight of the weighting can be determined on the basis of the recovery rate R. That is, data with a low recovery rate R is weighted relatively light. The smoothed elasticity ε_{c}(x, y) can be calculated according to expression (7) by using the recovery rate R(x, y) pertaining to the elasticity ε(x, y) at the coordinates (x, y), for example.

Σ_{c}(x,y,t)=Σ_{(-M≦m≦M)}Σ_{(-N≦n≦N)}(R(x+m,y+n,t)ε(x+m,y+n,t))/Σ_{(-M≦m≦M)}Σ_{(-N≦n≦N)}(R(x+m,y+n,t)ε(x+m,y+n,t))R(x+m,y+n,t) (7)

Likewise, the elasticity ε can be smoothed in the direction of a time axis. That is, an elasticity ε_{d}(x, y, t) smoothed in the direction of the time axis at the coordinates (x, y, t) is calculated according to the following expression (8).

ε_{d}(x,y,t)=Σ_{(-T≦k≦0)}(R(x+m,y+n,t)ε(x+m,y+n,t))/Σ_{(-T≦k≦0)}R(x,y,t+k) (8)

These spatial smoothing and smoothing in the direction of the time axis may be selected and executed separately or together.

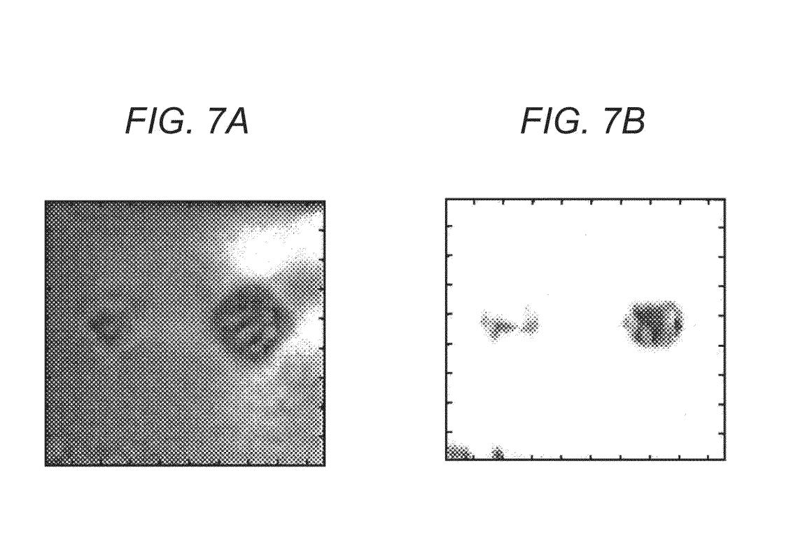

FIGS. 7A and 7B are diagrams illustrating display examples of an elasticity display image according to the ultrasound diagnostic apparatus U of the present embodiment.

FIG. 7A is a diagram illustrating the distribution of the elasticity, while FIG. 7B is a diagram illustrating the distribution of the recovery rate R pertaining to calculation of the elasticity.

The elasticity display image displays the elasticity within a two dimensional surface in color or gray scale. The dynamic range is adjusted to indicate in this case that a darker color corresponds to smaller elasticity and lower recovery rate (high elasticity coefficient and low accuracy). The aforementioned smoothing is performed on the elasticity display image. This way of display allows the user to easily notice the detection of a structure having a different degree of elasticity from the surrounding against the same pressure.

When the interior of the structure has a minute structure or is nonuniform, one can grasp the whole structure by many reflected waves generated in the interior of the structure. The recovery rate R is high in this case because the magnitude of the elasticity ε is calculated accurately for the whole structure. It is thus assumed that the recovery rate R is high in a region with a small elasticity ε in the left half range of FIGS. 7A and 7B and a region with a large elasticity ε in the upper right range, and that these elasticities ε (Young's modulus E) are calculated accurately.

When the interior of the structure is uniform or hollow, on the other hand, a boundary of the structure is detected at upper and lower ends of the structure by the reflected wave, but the accuracy of the magnitude of the calculated elasticity ε is decreased because the reflected wave from the interior is weak. Therefore, as indicated in a range near the center of the right side of FIGS. 7A and 7B, a region with a small elasticity ε is detected but, for a region in which the recovery rate R is low, the user can obtain the result while taking into consideration a low degree of accuracy of the result.

The ultrasound diagnostic apparatus U of the present embodiment as described above includes the image processing unit 16 that is the signal processing device which calculates the elasticity ε of the object resulting from the change in the compressed state by using the stretched waveform of ultrasound reflected from the object in the stretched state and the compressed waveform of ultrasound reflected from the object compressed by the predetermined pressure.

The processing control unit 16a of the image processing unit 16 calculates the phase difference F(t) between the stretched waveform r(t) and the approximated waveform c(t) associated with the compressed waveform s(t) at each elapsed time t, and calculates the elasticity εₖ by calculating the angular frequency difference εₖω₀ corresponding to the magnitude of the elasticity ε and the initial phase difference δₖ corresponding to the positional deviation between the stretched waveform r(t) and the approximated waveform c(t) according to the correlation between the plurality of elapsed times t and the corresponding phase difference F(t).

The elasticity ε and initial phase difference δ are calculated at the same time by directly using a linearity formed by small compression or stretch for which the linearity of the change in frequency holds, whereby the calculation of the elasticity ε using the discrete value in the measurement is less affected by a calculation error as compared to the related art using differentiation and integration. Unlike the calculation method of the related art, the elasticity ε and the initial phase difference δ are included in separate parameters as the slope and the intercept, respectively, so that the value of the elasticity ε is less affected by the initial phase difference δ. As a result, the calculation error of the elasticity can be reduced by the easy processing without increasing a cost of hardware resources such as an increase in speed of a CPU as well as an increase in size or addition of a memory. Moreover, there can be reduced the effect of a non-linear error more likely to occur in the calculation method of the related art when the elasticity ε is large.

Moreover, the processing of finding the elasticity εₖ is repeated by further stretching and phase-shifting the approximated waveform c(t) by the elasticity εₖ and initial phase difference δₖ calculated by the predetermined condition to update the approximated waveform, and using the updated approximated waveform c(t) and the stretched waveform r(t) to calculate the phase difference F(t) and calculate the angular frequency difference εₖω₀ and the initial phase difference δₖ. When the predetermined condition is satisfied, the elasticity ε is calculated on the basis of the cumulative value of the angular frequency difference εₖω₀ pertaining to the stretch of the compressed waveform s(t) and the approximated waveform c(t).

The more accurate elasticity ε can thus be found asymptotically. The processing can be repeated until the elasticity ε found in this way converges to a preferable level, and thus can be performed until the accurate elasticity ε is calculated without increasing unnecessary processing.

The aforementioned elasticity calculation processing is employed as the signal processing method of the echo, so that the elasticity ε can be calculated and output promptly and accurately while keeping down the processing load of the processing control unit 16a functioning as a control unit of the signal processing device and that appropriate information can be provided promptly while less affected by proficiency of a laboratory technician or doctor who is the user.

When the predetermined condition is satisfied, a correlation coefficient between the stretched waveform r(t) and the approximated waveform c(t) is calculated to be the recovery rate R. This allows the user to further obtain accuracy information pertaining to signal intensity or the like that cannot be known when the elasticity ε alone is calculated.

The output display unit 19 displays the calculated elasticity ε as well as the calculation accuracy thereof based on the recovery rate R thereon. This allows the user to efficiently obtain the elasticity ε and the accuracy thereof.

The image processing unit 16 performs noise removal by smoothing the space distribution of the calculated elasticity ε. The noise removal is performed by the weighted average of the magnitude of elasticity at each point ε(x+m, y−π) in the predetermined range {x+m, y−π: −M≦m≦M, −N≦n≦N} set for the elasticity ε(x, y) at the coordinates (x, y). The weight is determined on the basis of the recovery rate R(x+m, y−n). As a result, the accuracy of the space distribution associated with smoothing is not reduced unduly by treating the inaccurate data lightly, as compared to simple smoothing.

In calculating the phase difference F(t), the range of the elapsed time t for which the phase difference is calculated is changed according to the angular frequency difference εω₀ and/or the phase shift δ between the stretched waveform r(t) and the approximated waveform c(t). As a result, the number of data points can be increased to increase the calculation accuracy by calculating the phase difference F(t) with the elapsed time t having a narrow range when the elasticity ε and phase shift δ are so large that the foldback is more likely to occur in the middle of F(t) calculated, and calculating the phase difference F(t) with the elapsed time t having a wide range after the elasticity ε and phase shift δ are so reduced that the foldback is less likely to occur.

The repeat calculation is discontinued when the angular frequency difference εω₀ between the stretched waveform r(t) and the approximated waveform c(t) is smaller than or equal to the predetermined reference value, whereby the elasticity ε can be calculated accurately without unnecessarily requiring a long time.

The optimal angular frequency difference εω₀ and phase shift δ are calculated by the least squares method on the basis of the primary correlation concerning the phase difference F(t) and the corresponding elapsed time t, whereby the elasticity ε can be calculated easily and accurately.

The ultrasound diagnostic apparatus U of the present embodiment includes the ultrasound probe 2 transmitting/receiving the ultrasound and the ultrasound diagnostic apparatus body 1 including the image processing unit 16 that serves as the aforementioned signal processing device, so that the echo received by using the ultrasound probe 2 is processed near real-time to be able to promptly generate the data with low load and small error.

Note that the present invention is not limited to the aforementioned embodiment but can be modified in various ways.

While the aforementioned embodiment has described the example where the elasticity in a biological tissue is found by medical equipment, an object for which the elasticity is calculated is not limited to the biological tissue. The present invention can be used as appropriate for building structures and various products having a small structure as long as it is adapted to properly apply pressure to the object in the interior.

While there has been described in the aforementioned embodiment that the pressure applied to the object in the interior is changed by changing the pressure applied from the ultrasound probe 2, the pressure may be applied in a different manner. There may be used, for example, a technique (ARFI: acoustic radiation force impulse) of acquiring the echo by transmitting a strong sound wave for pressurization concurrently with the ultrasound for examination transmitted from the ultrasound probe 2.

The elasticity and Young's modulus are calculated on the precondition that the one dimensional compression is performed, in the aforementioned embodiment, but the calculation may be performed while considering compression and stretch in a three dimensional direction, namely in a plane perpendicular to the compression direction, according to the physical property of the object.

While the pressure is changed by pressing the ultrasound probe 2 against the object in the aforementioned embodiment, tension force may instead be applied depending on the object.

The recovery rate is calculated and output as a separate image in the aforementioned embodiment, but may be displayed as a numerical value or graph in a designated region or may be displayed in color along with the elasticity.

While the compressed waveform s(t) is stretched on the basis of the elasticity ε to approach the stretched waveform r(t) in the aforementioned embodiment, the stretched waveform r(t) can be compressed on the basis of the elasticity ε to approach the compressed waveform s(t) as well. When the stretched waveform r(t) and the compressed waveform s(t) are received alternately as described above, the stretch or compression may be performed alternately while aligning either the waveform acquired first or second with the other waveform as a fixed manner.

The output destination is not limited to the display screen of the output display unit 19 but may be an external device or external display. The data may also be directly output to a print output or output not as the image data but as numerical data to the external device.

Normally, the noise removal is performed to allow an image to be recognized more easily, but an image not objected to noise removal may be displayed as well. The noise removal can be performed not only by the weighted average but performed by using or using in combination another method based on an appropriate window setting.

In calculating the elasticity at each point, the range of the elapsed time t used in the calculation is widened according to the angular frequency difference and initial phase difference being set in the aforementioned embodiment, but the range may be changed while depending on either one of the differences.

Moreover, the image processing unit 16 of the present embodiment may be provided independently of the ultrasound probe 2 or another portion of the ultrasound diagnostic apparatus body 1. That is, the image processing unit may be provided as a dedicated signal processing device. The signal processing of the present invention being implemented by normal software processing, software may be installed to a computer such as a normal PC so that a control unit (CPU) of the computer executes the processing by using input waveform data.

Specific configurations, content of processing and procedures illustrated in the aforementioned embodiment can be modified as appropriate without departing from the spirit of the present invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. A signal processing device which uses a first signal waveform of ultrasound reflected from an object in a first compressed state and a second signal waveform of ultrasound reflected from the object in a second compressed state and calculates an elasticity of the object affected by a change in a compressed state, the device comprising:
a phase difference calculation unit which calculates a phase difference component in each time between the first signal waveform and the second signal waveform;
a correlation calculation unit which calculates an elasticity pertaining to a difference in angular frequencies between the first signal waveform and the second signal waveform and an initial phase difference according to a correlation between the each time and the phase difference component in the each time; and
an elasticity calculation unit which calculates the elasticity on the basis of the difference in angular frequencies.

2. A signal processing device which uses a first signal waveform of ultrasound reflected from an object in a first compressed state and a second signal waveform of ultrasound reflected from the object in a second compressed state and calculates an elasticity of the object affected by a change in a compressed state, the device comprising:
an approximated waveform generation unit which compresses or stretches and phase-shifts the second signal waveform by an elasticity and an initial phase difference related to a difference in angular frequencies being set and generates an approximated signal waveform approximated to the first signal waveform;
a phase difference calculation unit which calculates a phase difference component in each time between the first signal waveform and the approximated signal waveform;
a correlation calculation unit which calculates the elasticity and the initial phase difference between the first signal waveform and the approximated signal waveform according to a correlation between the each time and the phase difference component in the each time;
a repeat determination unit which updates the approximated signal waveform by compressing or stretching and phase-shifting the approximated signal waveform by using the elasticity and the initial phase difference calculated by the correlation calculation unit until a predetermined condition is satisfied, and uses the updated approximated waveform and the first signal waveform to repeat processing by the phase difference calculation unit and the correlation calculation unit; and
an elasticity calculation unit which calculates the elasticity on the basis of a cumulative value of the elasticity related to the compression repeatedly performed.

3. The signal processing device according to claim 2, further comprising a recovery rate calculation unit which calculates a recovery rate indicating a correlation between the first signal waveform and the approximated signal waveform when the predetermined condition is satisfied.

4. The signal processing device according to claim 3, further comprising a display unit and a display control unit which causes the display unit to display the elasticity and calculation accuracy of the elasticity based on the recovery rate.

5. The signal processing device according to claim 3, further comprising a noise removal unit which smoothes a space distribution of the elasticity calculated, wherein the noise removal unit calculates magnitude of the smoothed elasticity in each spatial position by a weighted average of magnitude of the elasticity in each of a plurality of positions within a predetermined range corresponding to the spatial position, and a weight of the weighted average is determined on the basis of magnitude of the recovery rate in each of the plurality of positions.

6. The signal processing device according to claim 2, wherein the phase difference calculation unit changes a width of the time pertaining to the calculated phase difference component according to at least one of the elasticity and the initial phase difference between the first signal waveform and the approximated signal waveform.

7. The signal processing device according to claim 2, wherein the repeat determination unit sets, as the predetermined condition, a case where the elasticity between the first signal waveform and the approximated signal waveform equals a predetermined reference value or smaller.

8. The signal processing device according to claim 1, wherein the correlation calculation unit calculates an optimal value of the elasticity and the initial phase difference by a least squares method using the elasticity and the initial phase difference as parameters.

9. An ultrasound diagnostic apparatus comprising:
an ultrasound probe which transmits/receives ultrasound; and
the signal processing device according to claim 1.

10. A signal processing method which uses a first signal waveform of ultrasound reflected from an object in a first compressed state and a second signal waveform of ultrasound reflected from the object in a second compressed state and calculates an elasticity of the object affected by a change in a compressed state, the method comprising:
a phase difference calculation step which calculates a phase difference component in each time between the first signal waveform and the second signal waveform;
a correlation calculation step which calculates an elasticity and an initial phase difference between the first signal waveform and the second signal waveform according to a correlation between the each time and the phase difference component in the each time; and
an elasticity calculation step which calculates the elasticity on the basis of the elasticity.

11. The signal processing device according to claim 2, wherein the correlation calculation unit calculates an optimal value of the elasticity and the initial phase difference by a least squares method using the elasticity and the initial phase difference as parameters.

12. An ultrasound diagnostic apparatus comprising:
an ultrasound probe which transmits/receives ultrasound; and
the signal processing device according to claim 2.
